(19) United States
(12) Patent Application Publication
(10) Pub. No.: US 2020/0085854 A1
(43) Pub. Date: Mar. 19, 2020

(54) COMPOSITION FOR USE IN THE TREATMENT OF EXTRAOESOPHAGEAL GASTRIC REFLUX SYMPTOMS

(71) Applicant: DRUGS MINERALS AND GENERICS ITALIA S.R.L. IN FORMA ABBREVIATA D.M.G. ITALIA S.R.L., Pomezia RM (IT)

(72) Inventor: Luigi MERCURI, Pomezia RM (IT)

(21) Appl. No.: 16/617,368

(22) PCT Filed: May 18, 2018
(86) PCT No.: PCT/IB2018/053514

§ 371 Date: Nov. 26, 2019

(30) Foreign Application Priority Data
May 26, 2017 (IT) 102017000057635

(51) Int. Cl. A61K 31/728; A61K 31/734; A61K 47/02; A61K 47/36

(52) U.S. Cl. CPC A61K 31/728; A61K 31/734; A61K 9/0043; A61K 47/36; A61K 47/02

(57) ABSTRACT

The present invention relates to a liquid mixture or solid mixture, in powder or granules, comprising or, alternatively, consisting of alginic acid and hyaluronic acid, or a salt thereof, for use in the treatment, in particular in the curative treatment, of extraoesophageal symptoms or disorders caused or provoked by gastroesophageal reflux (GERD) or by a partial reflux of gastric contents, such as, for example, the partial upflow of pepsin, hydrochloric acid, gastric juices or acidic gastric vapours from the stomach and wherein said disorders or symptoms manifest themselves in an extraoesophageal region. The present invention further relates to a liquid composition or solid composition, in powder or granules, which comprises said liquid or solid mixture comprising or, alternatively, consisting of alginic acid and hyaluronic acid, or a salt thereof, and optionally pharmaceutical or food grade additives or excipients, for use in the treatment, in particular in the curative treatment, of extraoesophageal disorders or symptoms that manifest themselves in the extraoesophageal region represented by the eye or eyeball and/or periocular region in patients affected by gastroesophageal reflux (GERD) or by a partial reflux of gastric contents, such as, for example, the partial upflow of pepsin, hydrochloric acid, gastric juices or acidic gastric vapours from the stomach.

The present invention relates to a liquid mixture or solid mixture, in powder or granules, comprising or, alternatively, consisting of alginic acid and hyaluronic acid, or a salt thereof, for use in the treatment, in particular in the curative treatment, of extraoesophageal disorders or symptoms caused or provoked by gastroesophageal reflux (GERD) or by a partial reflux of gastric contents, such as, for example the partial upflow of pepsin, hydrochloric acid, gastric juices or acidic gastric vapours from the stomach, and wherein said disorders or symptoms manifest themselves in an extraoesophageal region.

Furthermore, the present invention relates to a liquid composition or solid composition, in powder or granules, which comprises said liquid or solid mixture comprising or, alternatively, consisting of alginic acid and hyaluronic acid, or a salt thereof, and optionally pharmaceutical or food grade additives or excipients, for use in the treatment, in particular in the curative treatment, of extraoesophageal disorders or symptoms that manifest themselves in an extraoesophageal region represented by the eye or eyeball and/or periocular region in patients affected by gastroesophageal reflux (GERD) or a partial reflux of gastric contents, such as, for example, the partial upflow of pepsin, hydrochloric acid, gastric juices or acidic gastric vapours from the stomach.

In the context of the present invention, the term “composition” means a pharmaceutical composition or a composition for a medical device (briefly, the composition(s) of the present invention).

In otorhinolaryngology, the term “extraoesophageal reflux” identifies an irritative phenomenon caused by contact with the gastric acid that flows up to the larynx and beyond. In fact, reflux very frequently manifests itself only with these symptoms and not with the classic and popular “heartburn” due to GERD.

In this regard, it would perhaps be more correct to distinguish between two different nosological entities: classic gastroesophageal reflux (characterised by the typical symptom of retrosternal burning) and extraoesophageal reflux (in which the main symptoms are for example a dry cough, dysphonia, pharyngeal bolus). The latter phenomenon, contrary to what happens in classic reflux, is characterised by laryngeal irritation provoked by the reflux of gastric acids which end up affecting the upper respiratory tract. Although in some cases the two entities may coexist, much more frequently this does not occur. Patients with extraoesophageal symptoms due to reflux, as is logical to expect given the nature of the symptoms reported, much more frequently undergo an otolaryngologic examination rather than a gastroenterological examination. For this reason, ENT specialists are not infrequently the first to diagnose reflux in a subject who had been unaware until then of being affected by this disorder. Contrary to what happens in “classic reflux”, where reflux episodes are prevalently concentrated during the night, in the case of extraoesophageal reflux it has been demonstrated that the episodes occur throughout the whole 24-hour period.

Gastroesophageal reflux is a paraphysiological condition or disease characterised by the upflow of gastric contents, which comprises, for example, pepsin, hydrochloric acid, gastric juices and acidic gastric vapours, from the stomach to the oesophagus. After being swallowed, ingested food normally passes through a canal, the oesophagus, which conveys the alimentary bolus from the mouth to the inside of the stomach; here the strongly acidic environment enables the digestion of food, whose absorption takes place in the intestine.

The cardia is the anatomic region joining the oesophagus and stomach and is normally situated in the abdomen 2 to 4 cm below the diaphragm. Anatomically, the cardia is today considered part of the stomach. In the cardia we see the transition between the oesophageal and gastric mucosa.

Immediately upstream of the cardia, the circular muscle fibres of the oesophagus acquire a sphincteric action, in the sense that in rest conditions they remain contracted, whereas they relax during belching, the descent of food into the stomach and its return during vomiting; in other phases of digestive processes, by contrast, this functional sphincter should usually remain closed and contracted to prevent the acidic gastric contents of the stomach from rising up into the oesophagus and irritating the inner walls thereof. The sphincter we are talking about is known as the cardiac sphincter, gastroesophageal sphincter, lower oesophageal sphincter (LES) or cardiac valve.

In patients affected by disorders or symptoms or pathologies due to gastroesophageal reflux—GERD, there is a cardiac incapacity, more or less transitory, such as to render possible a reverse path of gastric contents. In patients with GERD, part of the gastric contents rises up from the stomach to the oesophagus, which is not provided with systems of protection against, for example, hydrochloric acid or gastric juices or against the acidic gastric vapours produced by the stomach. Gastric juice is a secretion produced by the internal mucosa of the stomach and containing mucus, salts, water, digestive enzymes (e.g. pepsin) and hydrochloric acid. The pH of gastric contents or gastric juice is very low, but may nonetheless vary, for example, from 1 to 2.

In GERD patients, the upflow, even in very small amounts, of gastric contents from the stomach to the oesophagus thus results in irritation of the epithelium of the oesophagus, which causes a burning sensation in a retrosternal position and pain upon swallowing, as well as an increase in caries (due to the corrosion of tooth enamel by gastric acids), retching after the intake of food, sensations of acidity in the upper part of the oesophagus and in the pharynx (particularly frequent when one assumes a horizontal position after eating, which facilitates the process of upflow of gastric contents into the oesophagus).

Gastroesophageal reflux is very common, sometimes being transient, and sometimes a symptom of a much more serious pathology. The most common causes may be obesity, diabetes mellitus, conditions of increased gastric secretion, pregnancy, smoking, alcohol, hiatal hernia (a congenital condition in which the oesophageal hiatus, the opening of the oesophagus into the stomach, is in an anomalous position that favours the upflow of gastric contents).

The pharynx is a canal that puts the throat in communication with the oesophagus. It has a muscular-mucosal structure and represents both the first section of the digestive tract—it receives the alimentary bolus from the mouth through swallowing—and a part of the upper respiratory tract: air coming from the nose is introduced into the pharynx, and from the pharynx it is introduced into the larynx. Therefore, the alimentary canal and respiratory tract both converge in the pharynx and then continue, respectively, in the oesophagus and in the larynx. The pharynx, about 15 centimetres long, runs behind the nasal cavities, mouth and larynx and extends vertically from the skull base to the sixth cervical vertebra. The pharynx is generally divided into three sections: the rear portion of the nasal airways (rhinopharynx), the so-called throat (oropharynx) and the laryngeal part (laryngopharynx). The rhinopharynx and oropharynx are separated by a specific portion of the palate: the soft palate (which represents the extension of the hard palate). The nasal cavities communicate with the pharynx by means of the choanae, with the mouth by means of the isthmus of the fauces (i.e. the narrow opening that represents the transition from the oral cavity to the pharyngeal one delimited from above by the free margin of the soft palate, by two pairs of folds of the mucosa at the sides and by the base of the tongue) and with the larynx by means of the laryngeal orifice. All of these communications are made at the front wall of the organ, which therefore appears in large part incomplete. There are two main functions of the pharynx: i) as the first section of the digestive tract, the pharynx puts the mouth in communication with the oesophagus, enabling the passage of the alimentary bolus through swallowing, and ii) as part of the upper respiratory tract, the pharynx enables the passage of air from the nasal cavities to the larynx.

In addition to disorders in the region of the oesophagus, patients affected by gastroesophageal reflux may also suffer from disorders (discomfort or mild complaints), symptoms (manifestation of a pathological state or even a series of phenomena with which a disease manifests itself) or pathologies (diseases) in extraoesophageal regions such as the pharynx, larynx, oropharynx, rhinopharynx, eye or eyeball and periocular region (which is situated around the eye or regards the area surrounding the eye, also including the lacrimal apparatus).

Disorders or symptoms similar to those experienced in an extraoesophageal region may also occur in individuals who are not affected by gastroesophageal reflux, the latter, as it is generally defined, resulting from the upflow, for example, of acidic gastric vapours from the stomach to an extraoesophageal region.

The reflux of gastric contents, such as, for example, the upflow of pepsin, hydrochloric acid, acidic gastric vapours or gastric and/or duodenal juices beyond the oesophagus into the larynx, oropharynx and nasopharynx is defined as a laryngopharyngeal disorder or “laryngopharyngeal reflux disease” (LPRD).

Although “laryngopharyngeal reflux disease” (LPRD for short) was initially considered as an extension of gastroesophageal reflux disease, it has recently come to be considered a disorder or pathology in itself, in particular in subjects of paediatric age (from birth to the beginning of puberty, generally between 12 and 14 years), since as a consequence it is able to give rise, locally, to pathological forms.

The lacrimal apparatus is part, together with the eyelids and conjunctiva, of the protective apparatus of the eye and consists of the lacrimal glands and lacrimal ducts, comprising, for example, the nasolacrimal duct.

It has been hypothesised that gastric acids are present in the nasolacrimal ducts of subjects affected by LPRD (Magliulo, G. et al. Medical Hypotheses 2013, 80, 129-130). Iannella, G. et al. (Int. J. Pediatr. Otorhinolaringol. 2015, 79, 2312-2315) found the presence of pepsin in the lacrimal secretions of children with LPRD. However, pepsin has been found to be present in only 20% of children diagnosed with LPRD. Furthermore, since the concentration detected has been deemed insufficient to give rise to inflammatory activity in the nasolacrimal duct and conjunctival precorneal cavity, the presence of pepsin has not been considered up to now as being unequivocally correlatable to the total number of reflux episodes or the type thereof.

As a consequence of the foregoing, anyone suffering from LPRD may also be subject to the so-called “dry eye syndrome”, i.e. an ocular pathology consisting in a quantitative reduction and/or qualitative alteration of the tear film, which mainly has a function of wetting the ocular surface. This syndrome is one of the most frequent pathologies in ophthalmology and its incidence is high in the middle-aged and elderly populations, increasing with age.

The definition of dry eye refers to a qualitative or quantitative alteration of the tear film, which is no longer able to keep the anterior surface of the eye healthy and protected. Tears have the task of performing fundamental functions: i) nutrient: tears make it possible to ensure a correct intake of oxygen and nutrient substances for a sufficient turnover of ocular surface cells;

ii) antimicrobial: the presence of antibodies and enzymes ensures a powerful defensive action against external aggressions; and
iii) cleaning, lubricant and optical: every time alterations occur in the quantity/quality of tears, these particular characteristics are lost as a consequence, thus exposing the eye to greater friction (determined by eyelid movement) and a higher risk of infection.

Another condition that can be linked to LPRD is conjunctivitis, i.e. an inflammation that affects the conjunctiva, or the membrane that lines the eyelid, and the cornea, the white part of the eye, giving rise to the classic characteristic of red eyes. Conjunctivitis may affect adults, but also children and infants.

One object of the present invention, therefore, is to provide mixtures or formulations or preparations or compositions for topical, i.e. not systemic, use, which are capable of treating disorders of the eye, eyeball and/or periocular region in a preventive and/or curative manner, said disorders being linked to or derived from or connected to extraoesophageal reflux or laryngopharyngeal reflux or, in general, the upflow of gastric contents or acidic gastric vapours from the stomach into an extraoesophageal region.

Another object of the present invention is to provide a composition for use in said preventive and/or curative treatment which is effective, stable, easy to administer/apply and which, once applied, is free of side effects; for example, it will not provoke irritation, burning or stickiness in the eye and/or periocular region.

These objects, and still others that will become apparent from the detailed description that follows, are achieved by the mixture and composition of the present invention thanks to the technical features claimed in the appended claims.

The present invention relates to a composition (C) comprising or, alternatively, consisting of:

i. a mixture which comprises or, alternatively, consists of the following compounds:
a) alginic acid, or a salt thereof;
b) hyaluronic acid, or a salt thereof; and, optionally,
   ii. at least one excipient or additive compatible with pharmaceutical use, for use in the curative and/or preventive treatment of disorders and symptoms caused or provoked, directly or indirectly, by gastroesophageal reflux and/or laryngopharyngeal reflux and/or in general by the upflow of gastric contents or acidic gastric vapours from the stomach and wherein said disorders or symptoms manifest themselves in an extraoesophageal region, preferably in patients affected by GERD.

The present invention further relates to a pharmaceutical composition or a composition for a medical device based on said composition (C) in a liquid state.

The present invention further relates to a pharmaceutical composition or a composition for a medical device based on said composition (C) in a solid state, in water-soluble granules or powder.

Unless specified otherwise, the indication that a composition “comprises” one or more components means that other components can be present in addition to the one, or ones, specifically indicated, even if they are not necessarily indicated—that is to say, the composition can also contain exclusively such components—and the indication that a composition “consists” of given components means that the presence of other components is excluded.

The composition (C) of the present invention has application for use in the treatment of symptoms, disorders, pathologies or effects caused or provoked by the upflow of gastric contents, for example pepsin or acidic gastric vapours or gastric and/or duodenal juices, from the stomach into an extraoesophageal area, both in subjects affected by GERD and in subjects who do not suffer from GERD.

The composition (C) according to the present invention is preferably for use for the treatment of said symptoms, disorders, pathologies or effects linked to the upflow of gastric contents, for example pepsin or acidic gastric vapours or gastric and/or duodenal juices, from the stomach into an extraoesophageal area in subjects who are affected by or suffer from GERD.

In one aspect, the present invention relates to a composition (C) comprising or, alternatively, consisting of:

i. a mixture which comprises or, alternatively, consists of the following compounds:
a) alginic acid, or a salt thereof;
b) hyaluronic acid, or a salt thereof; and, optionally,
   ii. at least one excipient compatible with pharmaceutical use, for use in the curative and/or preventive treatment of symptoms, disorders, pathologies or effects caused or provoked, directly or indirectly, by gastroesophageal reflux and/or laryngopharyngeal reflux or the upflow of gastric contents, for example pepsin or acidic gastric vapours, from the stomach into an extraoesophageal region and then, through the nasolacrimal duct and conjunctival precorneal cavity, to the eye and/or into the periocular region, preferably in patients affected by GERD.

The composition (C) for use of the present invention has valid application for the administration, both in subjects affected by or suffering from GERD and in subjects who are not affected by or suffering from GERD, on the ocular surface or in the periocular area, in the oral cavity and in the nasal cavity.

The present invention relates to a composition (C), for use in the treatment, preferably curative treatment, of the ocular surface or the periocular area or of dry eye.

The present invention relates to a composition (C) for use in the treatment, preferably curative treatment, of red eye or ocular inflammation or periocular inflammation.

The present invention relates to a composition (C) for use in the treatment, preferably curative treatment, of conjunctivitis.

For the sake of clarity, the treatment based on the use of the composition according to the present invention is by application of the composition on the ocular or periocular area (for example by means of a formulation in drops or cream or ointment) and/or in the oral cavity (for example by means of a liquid or spray formulation) and in the nasal cavity (for example by means of a formulation in drops or spray form).

Compositions for systemic use, such as solutions or solid forms to be swallowed or administered by injection, are outside the scope of the present invention.

Within the scope of the present invention, “periocular area” means the part of a human or animal body that lies around the ocular surface as defined above, i.e. which surrounds the eye. As a non-limiting example, the periocular area, as understood here, includes the eyelids, eyelashes, skin of the periorbital area and all tissues connected to or near the ocular surface.

In the context of the present invention, the term “medical device” is used within the meaning according to Italian Legislative Decree no. 46 of 24 Feb. 1997, which corresponds to the definition provided by the World Health Organization and available at the URL http://www.who.int/medical_devices/full_deffinition/en/, i.e. this term indicates a substance or another product, used alone or in combination, intended by the manufacturer to be used in human beings for the purposes of diagnosis, prevention, monitoring, treatment or alleviation of disease, which product does not achieve its primary action, in or on the human body for which it is intended, by pharmacological or immunological means or through a metabolic process, but which may be assisted in its function by such means.

The composition (C) according to the present invention can be advantageously used also in the curative and/or preventive treatment of conjunctivitis both in subjects affected by or suffering from GERD and in subjects not affected by or suffering from GERD, preferably in subjects who suffer from gastroesophageal reflux (GERD).

Advantageously, the composition (C) according to the present invention makes it possible to treat, for curative and/or preventive purposes, disorders, symptoms and pathologies caused by gastroesophageal reflux and/or laryngopharyngeal reflux or the upflow of gastric contents or acidic gastric vapours from the stomach into an extraoesophageal region and which manifest themselves in the eye and/or in the periocular region without administering substances of a steroidal type, which may cause side effects, also severe ones, and may exacerbate the disorder due to gastroesophageal reflux, in addition to causing an increase in ocular pressure and increasing the risk of glaucoma.

The composition (C) according to the present invention preferably contains no active ingredients of a steroidal type.

Advantageously, the composition (C) according to the present invention makes it possible to treat, for curative and/or preventive purposes, disorders, symptoms and pathologies caused by gastroesophageal reflux and/or laryngopharyngeal reflux or the upflow of gastric contents or acidic gastric vapours from the stomach into an extraoesophageal region and which manifest themselves in the eye and/or in the periocular region more effectively than prior art compositions for the treatment of disorders, symptoms and pathologies which manifest themselves in the eye and/or in the periocular region.

In a first preferred embodiment, the mixture is made up of alginic acid, or a salt thereof, and hyaluronic acid, or a salt thereof, and the composition (C) according to the present invention which contains said mixture contains no active ingredients in addition to the ones present in said mixture and, optionally, pharmaceutically acceptable excipients and/or additives.

In another preferred embodiment, the composition (C) according to the present invention comprises or consists of a mixture comprising, in addition to component (a) alginic acid, or a salt thereof and component (b) hyaluronic acid, or a salt thereof, at least one other compound or active ingredient; said at least one other compound or active ingredient is preferably selected from:
Green tea (Camellia sinensis) extract, preferably glycerinic green tea extract, in an amount comprised from 0.1% to 5% by weight; more preferably in an amount comprised from 0.5% to 3.5% by weight; even more preferably in an amount comprised from 1% to 2% by weight, relative to the weight of the composition (C) and/or;
Rosmarinic acid (3-(3,4-dihydroxyphenyl)-2R-{[3-(3,4-dihydroxyphenyl)-prop-2E-enoyl]oxy}propanoic acid), preferably in an amount comprised from 0.01% to 3% by weight; more preferably in an amount comprised from 0.1% to 2% by weight; even more preferably in an amount comprised from 0.5% to 1% by weight, relative to the weight of the composition (C) and/or;
ethylenediaminetetraacetic acid (EDTA) or a salt thereof, preferably in an amount comprised from 0.05% to 0.3% by weight; more preferably in an amount comprised from 0.1% to 0.25% by weight; even more preferably in an amount comprised from 0.15 to 0.20 by weight, relative to the weight of the composition (C).

Alginic acid (CAS No. 9005-32-7) is a hydrophilic colloidal polysaccharide with a (mean) molecular weight comprised from about 50,000 Dalton to about 800,000 Dalton; preferably comprised from about 100,000 Dalton to about 600,000 Dalton; even more preferably comprised from about 200,000 to about 400,000, for example to about 240,000 Dalton (atomic mass units), which is obtained from seaweed. In the mixture and in the composition (C) according to the present invention, a) is a preferably an alginate salt, more preferably magnesium alginate.

Hyaluronic acid (CAS No. 9004-61-9) is a non-sulphated glycosaminoglycan having an unbranched polysaccharide chain deriving from the condensation of disaccharide units which are formed, in turn, by residues of glucuronic acid and N-acetylglucosamine, bonded together, alternatively, by β1→4 and β1→3 glycosidic bonds. Hyaluronic acid is thus also used in the form of a salt. The mixture contained in the composition (C) according to the present invention can comprise hyaluronic acid, or salts thereof, having a different origin and various ranges of molecular weights, and it can be linear or branched.

Within the scope of the present invention, the hyaluronic acid can be in acidic or salified form, for example as a sodium salt.

Preferably, but without limitation, the mixture contained in said composition (C) according to the present invention comprises linear or branched hyaluronic acid, or salts thereof, for example a sodium salt, with a (mean) molecular weight comprised between 200 and 5000 kDa; more preferably, it is comprised between 1000 and 3000 kDa; it is even more preferably comprised between 1500 and 2000 kDa.

The composition (C) according to the present invention can comprise a) and b), and optionally also other substances such as additives and excipients, in varying amounts and in different weight ratios to one another.

In the compositions (C) of the present invention: a) component a) is comprised from 0.05 to 5% by weight; it is preferably comprised from 0.2 to 2% by weight; even more preferably it is comprised from 0.4 to 0.8 by weight, relative to the total weight of the composition (C), and

b) component b) is comprised from 0.01 to 1% by weight; it is preferably comprised from 0.05 to 0.5% by weight; it is even more preferably comprised from 0.15 to 0.30 by weight, relative to the total weight of the composition (C).

It is understood that the present invention relates to the use of a) alginic acid, or a salt thereof, and b) hyaluronic acid, or a salt thereof, in the treatment of symptoms and pathologies caused by gastroesophageal reflux in an extraoesophageal area, preferably in the eye and/or in the periocular region, both when a) and b) are administered simultaneously, i.e. in a mixture in the same composition, and when a) and b) are administered to a subject in any order, in close sequence over time in two distinct compositions.

In one embodiment, the composition (C) according to the present invention is in the form of a liquid preparation, such as, for example, an aqueous or oily collyrium, suspension or eye drops, a semisolid form, such as, for example, an unguent, ointment, gel or cream, or a solid form, such as, for example, a powder, capsule, tablet, compress, ocular insert and the like.

The composition (C) according to the present invention can comprise at least one excipient for pharmaceutical use, or commonly used in medical devices, included among the ones known to the person skilled in the art and utilizable in ophthalmic formulations, or for topical nasal or oral use, in liquid, semisolid or solid form.

As a non-limiting example, the excipients that can be included as a component ii. in the composition (C) according to the present invention comprise preservatives, antioxidants, stabilisers, thickeners, rheology modifiers, biocides, colourants, pH buffers and the like.

In a preferred embodiment, the composition (C) according to the present invention is in a form suitable for ocular administration, preferably as an aqueous or oily collyrium, suspension or eye drops, an unguent, ointment, gel, cream or eye insert.

In a preferred embodiment, the composition (C) according to the present invention is in a form suitable for administration in the oral cavity, for example as an oral spray, syrup, solution and like forms, more preferably in the form of an oral spray.

In a preferred embodiment, the composition (C) according to the present invention is in a form suitable for administration in the nasal cavity, for example as an oral spray, drops, powder, lavage, isotonic solution, aerosol and like forms, more preferably in the form of a nasal spray.

In one embodiment, the composition (C) or medical device according to the present invention is in the form of a collyrium, comprising boric acid and/or sodium tetraborate, preferably, each independently of the other, in an amount comprised from 0.1 to 1% by weight out of the total weight of the pharmaceutical composition or medical device.

In a particularly preferred embodiment, the composition according to the present invention is in the form of an ophthalmic solution (e.g. collyrium) and comprises, per 500 g of composition:

| Component | % by weight/total weight | Weight (g) |
|---|---|---|
| Magnesium alginate | 0.20 | 1 |
| Hyaluronic acid | 0.15 | 0.75 |
| Glycerinic green tea extract | 1.00 | 5 |
| and, optionally, excipients come: | | |
| Sodium tetraborate | 0.20 | 1 |
| Boric acid | 0.70 | 3.5 |
| Sodium chloride | 0.24 | 1.2 |
| Water | 97.51 | 487.55 |

In a particularly preferred embodiment, the composition according to the present invention is in the form of a nasal spray and comprises per 200 g of composition:

| Component | % by weight/total weight | Weight (g) |
|---|---|---|
| Magnesium alginate | 0.20 | 0.4 |
| Hyaluronic acid | 0.10 | 0.2 |
| Glycerinic green tea extract | 1.00 | 2 |
| and, optionally, excipients such as: | | |
| Na phosphate dibasic | 0.40 | 0.8 |
| Na phosphate monobasic | 0.15 | 0.3 |
| Sodium chloride | 0.38 | 0.76 |
| EDTA | 0.10 | 0.2 |
| Microglicin 50 | 0.10 | 0.2 |
| Water | 97.57 | 195.14 |

In a particularly preferred embodiment, the composition according to the present invention is in the form of an oral spray and comprises per 900 g of composition:

| Component | % by weight/total weight | Weight (g) |
|---|---|---|
| Magnesium alginate | 0.20 | 1.8 |
| Hyaluronic acid | 0.10 | 0.9 |
| Glycerinic green tea extract | 1.00 | 9 |
| and, optionally, excipients such | 0.20 | 1.8 |
| as- Sodium benzoate | | |
| Potassium sorbate | 0.20 | 1.8 |
| Potassium hydrate | 0.022 | 0.2 |
| EDTA | 0.10 | 0.2 |
| Natural flavouring | 0.15 | 1.35 |
| Water | 93.03 | 837.25 |

### EXPERIMENTAL SECTION

The following examples provide practical embodiments of the invention, without intending to limit the object or scope thereof.

COMPOSITION 1: ophthalmic solution (collyrium) which comprises per 500 g of composition:

| Component | % by weight/total weight | Weight (g) |
|---|---|---|
| Magnesium alginate | 0.20 | 1 |
| Hyaluronic acid | 0.15 | 0.75 |
| Glycerinic green tea extract | 1.00 | 5 |
| and, optionally, excipients such as: | | |
| Sodium tetraborate | 0.20 | 1 |
| Boric acid | 0.70 | 3.5 |
| Sodium chloride | 0.24 | 1.2 |
| Water | 97.51 | 487.55 |

COMPOSITION 2: nasal spray which comprises per 200 g of composition:

| Component | % by weight/total weight | Weight (g) |
|---|---|---|
| Magnesium alginate | 0.20 | 0.4 |
| Hyaluronic acid | 0.10 | 0.2 |
| Glycerinic green tea extract | 1.00 | 2 |
| and, optionally, excipients such as: | | |
| Na phosphate dibasic | 0.40 | 0.8 |
| Na phosphate monobasic | 0.15 | 0.3 |
| Sodium chloride | 0.38 | 0.76 |
| EDTA | 0.10 | 0.2 |
| Microglicin 50 | 0.10 | 0.2 |
| Water | 97.57 | 195.14 |

COMPOSITION 3: oral spray which comprises per 900 g of composition:

| Component | % by weight/total weight | Weight (g) |
|---|---|---|
| Magnesium alginate | 0.20 | 1.8 |
| Hyaluronic acid | 0.10 | 0.9 |
| Glycerinic green tea extract | 1.00 | 9 |
| and, optionally, excipients such as: | | |
| Sodium benzoate | 0.20 | 1.8 |
| Potassium sorbate | 0.20 | 1.8 |
| Potassium hydrate | 0.022 | 0.2 |
| EDTA | 0.10 | 0.2 |
| Natural flavouring | 0.15 | 1.35 |
| Water | 93.03 | 837.25 |

Preferred embodiments FRn of the present invention are set forth below:

FR1. A composition comprising or, alternatively, consisting of:

i. a mixture which comprises the following components:
a) alginic acid, or a salt thereof;
b) hyaluronic acid, or a salt thereof; and, optionally,
   ii. at least one excipient compatible with pharmaceutical use, for use in the treatment of symptoms, disorders or effects caused or provoked by gastroesophageal reflux or laryngopharyngeal reflux or the upflow of acidic gastric vapours from the stomach into an extraoesophageal region, preferably in patients affected by GERD.

FR2. The composition for use according to FR1, wherein said composition is for administration, both in subjects affected by or suffering from GERD and in subjects who are not affected by or suffering from GERD, on the ocular surface or in the periocular area, the oral cavity and the nasal cavity.

FR3. The composition for use according to FR1 or FR2, wherein said composition is for use in the treatment, preferably curative treatment, of the ocular surface or the periocular area or of dry eye.

FR4. The composition for use according to FR3, wherein said composition is for use in the treatment, preferably curative treatment, of red eye or ocular inflammation or periocular inflammation.

FR5. The composition for use according to FR3, wherein said composition is for use in the treatment, preferably curative treatment, of conjunctivitis and the state of health of the conjunctiva.

FR6. The composition for use according to any one of the preceding embodiments FR1-FR5, wherein in said composition:

a) component a) is comprised from 0.05 to 5% by weight; it is preferably comprised from 0.2 to 2% by weight; it is even more preferably comprised from 0.4 to 0.8 by weight, relative to the total weight of the composition, and
b) component b) is comprised from 0.01 to 1% by weight; it is preferably comprised from 0.05 to 0.5% by weight; it is even more preferably comprised from 0.15 to 0.30 by weight, relative to the total weight of the composition.

FR7. The composition for use according to any one of the preceding embodiments FR1-FR6, wherein said component a) is an alginic acid; it is preferably alginic acid (CAS no. 9005-32-7), and has a (mean) molecular weight comprised from about 50,000 Dalton to about 800,000 Dalton; preferably comprised from about 100,000 Dalton to about 600,000 Dalton; even more preferably comprised from about 200,000 to about 400,000 Dalton, for example to about 240,000 Dalton (atomic mass units) and it is obtained from seaweed; said component a) is preferably an alginate salt, more preferably magnesium alginate.

FR8. The composition for use according to any one of the preceding embodiments FR1-FR7, wherein said component b) is a linear or branched hyaluronic acid, with a (mean) molecular weight comprised between 200 and 5,000 kDa; it is more preferably comprised between 1.000 and 3.00 kDa; it is even more preferably comprised between 1,500 and 2,000 kDa; said component b) is preferably a hyaluronate salt, more preferably a sodium salt.

FR9. The composition for use according to any one of the preceding embodiments FR1-FR8, wherein said composition comprises, in addition to component (a) alginic acid, or a salt thereof and component (b) hyaluronic acid, or a salt thereof, at least one other compound or active ingredient; said at least one other compound or active ingredient is preferably selected from:
Green tea (Camellia sinensis) extract, preferably glycerinic green tea extract in an amount comprised from 0.1% to 5% by weight; more preferably in an amount comprised from 0.5% to 3.5% by weight; even more preferably in an amount comprised from 1% to 2% by weight, relative to the weight of the composition (C) and/or;
Rosmarinic acid (3-(3,4-dihydroxyphenyl)-2R-{[3-(3,4-dihydroxyphenyl)-prop-2E-enoyl]oxy}propanoic acid), preferably in an amount comprised from 0.01% to 3% by weight; more preferably in an amount comprised from 0.1% to 2% by weight; even more preferably in an amount comprised from 0.5% to 1% by weight, relative to the weight of the composition (C) and/or;
ethylenediaminetetraacetic acid (EDTA) or a salt thereof, preferably in an amount comprised from 0.05% to 0.3% by weight; more preferably in an amount comprised from 0.1% to 0.25% by weight; even more preferably in an amount comprised from 0.15 to 0.20 by weight, relative to the weight of the composition (C).

FR10. The composition for use according to any one of the preceding embodiments FR1-FR9, wherein said composition is:
in liquid form; it is preferably an aqueous or oily collyrium, a suspension or eye drops;
in semi-solid form; it is preferably an unguent, an ointment, a gel or a cream;
in solid water-soluble form; it is preferably a powder, a capsule or a tablet.

1. A composition comprising or, alternatively, consisting of:
i. a mixture which comprises the following components:
a) alginic acid, or a salt thereof;
b) hyaluronic acid, or a salt thereof; and, optionally,
ii. at least one excipient compatible with pharmaceutical use, for use in the treatment of symptoms, disorders or effects caused or provoked by laryngopharyngeal reflux or the upflow of acidic gastric vapours from the stomach into an extraoesophageal region, or an eye disorder.

2. The composition according to claim 1, wherein said composition is for use in the treatment of symptoms, disorders or effects caused or provoked by gastroesophageal reflux.

3. The composition according to claim 1, wherein said composition is for administration, both in subjects affected by or suffering from GERD and in subjects who are not affected by or suffering from GERD, on the ocular surface or in the periocular area, the oral cavity and the nasal cavity.

4. The composition according to claim 1, wherein said composition is for use in the treatment, of the ocular surface or the periocular area or of dry eye.

5. The composition according to claim 4, wherein said composition is for use in the treatment of red eye or ocular inflammation or periocular inflammation.

6. The composition according to claim 4, wherein said composition is for use in the treatment of conjunctivitis and the state of health of the conjunctiva.

7. The composition according to claim 1, wherein in said composition:
a) component a) is comprised from 0.05 to 5% by weight, relative to the total weight of the composition, and
b) component b) is comprised from 0.01 to 1% by weight, relative to the total weight of the composition.

8. The composition according to claim 1, wherein said component a) is an alginic acid and has a (mean) molecular weight comprised from about 50,000 Dalton to about 800,000 Dalton and it is obtained from seaweed, or a salt thereof.

9. The composition according to claim 1, wherein said component b) is a linear or branched hyaluronic acid, with a (mean) molecular weight comprised between 200 and 5,000 kDa or salt thereof.

10. The composition according to claim 1, wherein said composition comprises, in addition to component (a) alginic acid, or a salt thereof and component (b) hyaluronic acid, or a salt thereof, at least one other compound or active ingredient; said at least one other compound or active ingredient is selected from:
Green tea (Camellia sinensis) extract in an amount comprised from 0.1% to 5% by weight, relative to the weight of the composition (C) and/or;
Rosmarinic acid (3-(3,4-dihydroxyphenyl)-2R-{[3-(3,4-dihydroxyphenyl)-prop-2E-enoyl]oxy}propanoic acid) in an amount comprised from 0.01% to 3% by weight, relative to the weight of the composition (C) and/or;
ethylenediaminetetraacetic acid (EDTA) or a salt thereof, in an amount comprised from 0.05% to 0.3% by weight, relative to the weight of the composition (C).

11. The composition according to claim 1, wherein said composition is:
in liquid form, preferably an aqueous or oily collyrium, a suspension or eye drops;
in semi-solid form, preferably an unguent, an ointment, a gel or a cream;
in solid water-soluble form, preferably a powder, a capsule or a tablet.

12. A method of treatment comprising:
administering a mixture which comprises the following components:
a) alginic acid, or a salt thereof;
b) hyaluronic acid, or a salt thereof;
to a patient in need thereof.

13. The method of claim 12, wherein the patient has a disorder associated with laryngopharyngeal reflux or the upflow of acidic gastric vapours from the stomach into an extraoesophageal region.

14. The method of claim 13, wherein the patient has GERD

15. The method of claim 12, wherein the patient has an eye disorder.

16. The method of claim 15, wherein the patient has red eye, ocular inflammation or periocular inflammation, or dry eye.

17. The method of claim 12, wherein the administration is to the eye.

18. The method of claim 12, wherein component a) is comprised from 0.05 to 5% by weight relative to the total weight of the composition, and component b) is comprised from 0.01 to 1% by weight relative to the total weight of the composition.

19. The method of claim 12, wherein the composition component a) is an alginic acid and has a (mean) molecular weight comprised from about 50,000 Dalton to about 800,000 Dalton—and it is obtained from seaweed, or a salt thereof.

20. The method of claim 12, wherein component b) is a linear or branched hyaluronic acid, with a (mean) molecular weight comprised between 200 and 5,000 kDa, or salt thereof.
